(19) United States
(12) Patent Application Publication
(10) Pub. No.: US 2026/0263012 A1
(43) Pub. Date: Sep. 10, 2026

(54) METHOD FOR DETERMINING AN INDIVIDUAL'S CONDITION IN RELATION TO REFERENCE CONDITIONS

(71) Applicant: GEODAISICS, Grenoble (FR)

(72) Inventors: Arnaud Attye, Grenoble (FR); Felix Renard, Grenoble (FR)

(73) Assignee: GEODAISICS, Grenoble (FR)

(21) Appl. No.: 19/473,150

(22) PCT Filed: Apr. 7, 2024
(86) PCT No.: PCT/EP2024/059415

§ 371 Date: Oct. 7, 2025

(30) Foreign Application Priority Data
Apr. 8, 2023 (FR) 2303553

(51) Int. Cl. A61B 5/00; A61B 6/00; G06T 7/00; G06T 7/73

(52) U.S. Cl. CPC A61B 5/7267; G06T 7/0014; G06T 7/74; A61B 6/5217; G06T 2207/20076; G06T 2207/20081; G06T 2207/30004

(57) ABSTRACT

The method for processing physiological features of an individual includes a) based on the physiological features of the individual (m_{i,test}), assigning coordinates in an input space (R) to the individual, with each coordinate being assigned a rank (i); b) associating each coordinate (x_{i,test}) of the individual, in the input space, with a latent space (Eₖ) belonging to a set of latent spaces, each latent space having a dimension smaller than the dimension of the input space; c) projecting each coordinate resulting from a) into the latent space associated with said coordinate, to obtain a latent vector (Y_{j,k}); d) applying a regression function (ƒₖ⁻¹, ƒ_{i,k}⁻¹) to each latent vector in order to estimate an input coordinate (\hat{x}_{i,test,k}) in the input space; and e) comparing (x_{i,test}−\hat{x}_{i,test,k} the input coordinates of the individual resulting from d) and a) and determining a score for the individual (z_{i,test}).

### TECHNICAL FIELD

The technical field of the invention relates to data processing for evaluating an individual's condition relative to reference conditions defined from a reference population. More specifically, the technical field concerns manifold learning techniques for classifying individuals from physiological data established for each of them.

### BACKGROUND ART

Biological or medical tests performed on patients are intended for use by practitioners to aid in diagnosis. A common practice is to compare physiological features determined for a patient with reference physiological features established for a reference population considered to be healthy.

The digitization of tests makes it possible to build large databases, for different types of tests, such as biological analysis results or data obtained through imaging or other types of diagnoctic measurements performed on a patient. These may include, for example, electrophysiological measurements such as ECG (electrocardiogram) or EEG (electroencephalogram).

Building databases makes it possible to determine reference physiological features with greater statistical accuracy. It also allows for better individualization of reference values: contextual data (age, weight, height, sex), or physiological data resulting from other types of examinations, may be taken into account to refine the reference physiological features.

Data projection into a latent space is a well-known method. For example, US2023/0022257 describes a method for filtering neural connections (neurological fibers) obtained by tractography. Filtering is performed by projecting an image into a latent space in which reference neural connections have been projected. The projection of the reference neural connections forms a cluster in the latent space. In the latent space, the projected connections closest to or within the cluster are considered reliable, while the others are rejected.

The publication Attyé et al “Tractlearn: a geodesic learning framework for quantitative brain bundles” describes the use of a dimensionality reduction algorithm using manifold learning, to analyse brain structures based on features resulting from magnetic resonance imaging (MRI).

### SUMMARY OF THE INVENTION

The inventors have improved the method described in the Attyé publication in order to enhanced the sensitivity and specificity of the analysis of physiological data measured on a patient.

A first object of the invention is a method for processing physiological data of an individual, each physiological data resulting from measurements taken on the individual, the method comprising the following steps:
a) based on the physiological data of the individual, assigning coordinates, known as input coordinates, in an input space to the individual, with each input coordinate being assigned a rank;
b) associating each input coordinate of the individual, in the input space, with a latent space, of a dimension smaller than the dimension of the input space;
c) projecting each input coordinate resulting from a) into the latent space associated with said coordinate, so as to obtain a latent coordinate of the individual in said latent space, and forming a latent vector in the latent space associated with said input coordinate;
d) applying a regression function to the latent vector resulting from c), so as to estimate an input coordinate, in the input space, for each rank, the regression function being associated with the latent space associated with the input coordinate;
e) for at least one rank, comparing the input coordinates of the individual resulting from d) and a) and determining a score for the individual based on the comparison.

The method may be such that
in b), the latent space associated with each input coordinate belongs to a set of latent spaces, the association of each input coordinate with a latent space being performed according to the rank of said coordinate;
in c), the latent coordinates in the same latent space form a latent vector;
d) involves applying a regression function to each latent vector resulting from c):

The method may be such that each latent space is determined from physiological data established on reference individuals from a reference population, according to the following steps:
i) assigning reference coordinates to each reference individual in the input space, each reference coordinate being established from physiological data resulting from a measurement taken on the reference individual;
ii) applying a dimension reduction algorithm to the reference coordinates of each reference individual, so as to define, for each reference individual, latent coordinates in a latent space, step ii) being implemented, during a first iteration, for all ranks and, during subsequent iterations, for ranks not selected in step v) of a previous iteration, the latent coordinates in the same latent space forming a latent vector;
iii) for each rank, applying a regression function to each latent vector in order to obtain an estimate of the input coordinate for said rank in the input space;
iv) comparing the input coordinates of each reference individual resulting from steps (i) and (iii) in order to obtain, for each rank, a relevance indicator;
v) taking into account a selection condition and selecting ranks for which the relevance indicator satisfies the selection condition;
vi) repeating steps ii) to v) for the ranks not selected in v), the repetition being carried out until an iteration termination criterion is reached.

According to one possibility, step c) involves applying a regression function to each latent vector in the latent space, step c) being such that two regression functions, applied to the same latent vector and allowing the estimation of two different input coordinates, are different.

During the first iteration, step ii) may include:
ii-a) based on the coordinates of the reference individuals in the latent space, selecting reference individuals representative of the reference population;
ii-b) repeating steps i) and ii) without taking into account the reference individuals selected in sub-step ii-b).

Sub-step ii-a) may include:
estimating a probability density from the latent vectors respectively associated with each reference individual;
for each reference individual, calculating a probability density value from the latent vector associated with the reference individual;
selecting reference individuals for which the probability density value is greater than a predetermined threshold.

The method may include, prior to step a), obtaining physiological data from the individual using at least one sensor.

The method may include:
forming a two-dimensional or three-dimensional image of the individual;
extracting physiological data from each image of the individual.

The image may result from an imaging modality such as MRI (Magnetic Resonance Imaging), computed tomography, or scintigraphy.

The physiological data may result from an analysis modality such as: electrocardiogram, electromyogram, electroencephalogram, magnetoencephalography, oximetry, or body gas composition analysis.

The method may include or be applied after analysis of a bodily fluid of the user, previously collected, with the physiological data of the individual resulting from the analysis of the bodily fluid.

A second object of the invention is a system for determining an individual's condition, the system comprising:
a sensor, or a set of sensors, configured to measure physiological data of the individual;
a processing unit configured to receive the physiological data measured by the sensor or set of sensors and to implement steps a) to e) of a method according to the first embodiment.

The invention will be better understood upon reading the description of the examples of embodiments presented below, in conjunction with the figures listed below.

### BRIEF DESCRIPTION OF THE DRAWINGS

FIG. 1 shows an example of a system suitable for implementing the invention.

FIG. 2 schematically illustrates the main steps for implementing a method according to the invention.

FIG. 3 illustrates an association of several latent spaces with input coordinates.

FIG. 4 shows a histogram of the value of a parameter of an estimator (size of the estimator window) for different features.

FIG. 5 represents, for different features studied (x-axis), the mean square error (y-axis) between the standardized feature xᵢ and its estimate \hat{x}_{i,k} calculated by regression.

FIG. 6 shows a selection of individuals based on latent vectors defined in a latent space.

### DETAILED DESCRIPTION OF PARTICULAR EMBODIMENTS

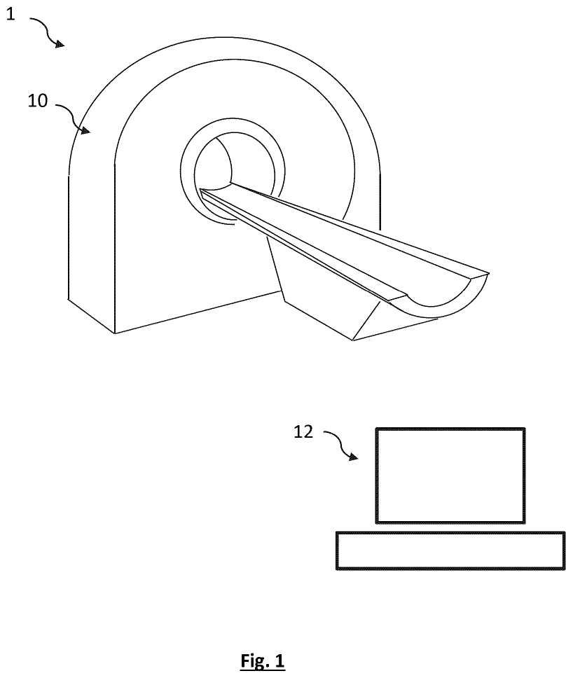

FIG. 1 shows an example of a system for implementing the invention. System 1 comprises a system 10 for acquiring physiological data from an individual. In this example, the acquisition system is an MRI system. The individual is a human being or animal. Physiological data refers to a value of a physiological parameter of the individual, which may vary depending on the individual's state of health. This may be data resulting from an image acquisition system or an electrophysiological measurement acquisition system (e.g., ECG—Electrocardiogram, EEG—Electroencephalogram, EMG—Electromyogram, MEG—Magnetoencephalography, oximetry, or any other analysis of a body gas, particularly a respiratory gas). It may also be data measured on a sample that has been previously taken from the patient. The sample may be, for example, a body fluid such as blood or urine. The data may be the concentration of a cell, protein, or other molecule of biological interest in the body fluid. When the measured data results from an analysis of a body fluid, the physiological data acquisition system may be a biological analysis machine.

System 1 includes a processing unit 12, programmed to receive the data measured by the acquisition system and implement the processing steps described below. The processing unit may be a computer or a remote cloud computing architecture.

One objective of the invention is to enable the identification, using measured physiological data, of a possible abnormality in the patient compared to a reference population. It is then up to a practitioner to determine the cause of the abnormality and any pathological condition of the individual. Thus, the method does determine a possible pathological condition of the individual based on a possible abnormality in the patient.

The physiological data of the individual, measured by the acquisition system 10 and processed by the processing unit 12, are preferably available in large numbers. The number of physiological data points is preferably greater than 5 or even 10. It is typically in the tens. As in the publication cited in the prior art, the method implements a manifold learning method using linear or nonlinear dimensionality reduction methods. Various methods of data dimensionality reduction are available. In general, a dimensionality reduction method allows the input coordinates in an input space (or real space) R of dimension I to be represented as coordinates in an output space E of dimension H, where I<H. The output space is usually referred to as the “latent space”. As previously indicated, the dimension of the input space R, may be several tens. The dimension of the output space E is preferably less than 10. The input coordinates, or features, are formed from the individual's physiological data. The input coordinates form a feature vector.

The dimension of the output space depends on the number of physiological features and their complexity. Preferably, it is less than 10 in order to facilitate calculations.

The transition from the input space R to the latent space E is performed by a projection function ƒ applied to the vector formed from the physiological features, known as the feature vector.

The transition from the latent space E to the input space R is performed by a backprojection function ƒ⁻¹ applied to a vector formed by the coordinates in the latent space.

The latent space E, as well as the projection and backprojection functions, are defined during a training phase, during which a set of reference physiological feature vectors is available. Each vector of reference physiological features contains data measured on reference individuals forming a reference population. The objective of dimensionality reduction is to obtain a representation of the reference physiological features in the latent space, the latter being of limited dimension compared to the input space.

Several methods of dimensionality reduction are known to those skilled in the art. For example, principal component analysis (PCA) is an unsupervised and linear method of dimensionality reduction. The basis of the latent space is formed by eigenvectors of the covariance matrix of the feature vectors. Nonlinear methods may be implemented, for example a multidimensional scaling (MDS) method, which preserves the distance values between all pairs of data in the input space. An example of an MDS method is the Sammon method.

Other nonlinear methods include the Isometric Feature Mapping (Isomap) method, which defines a transformation that preserves the geodesic distance between data points. Another type of method is the Uniform Manifold Approximation and Projection (UMAP) method.

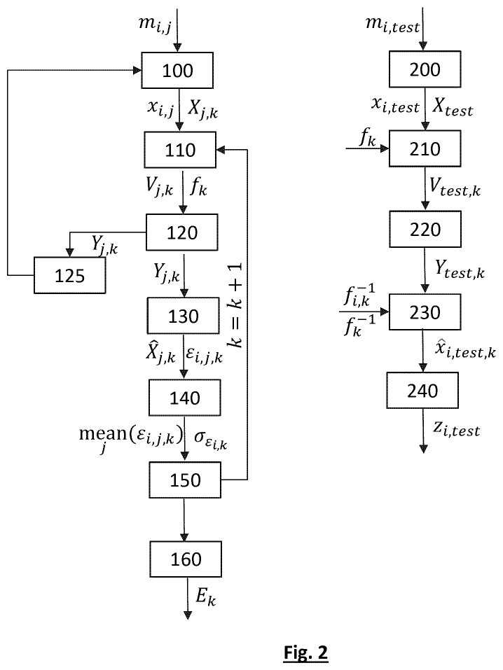

FIG. 2 schematically illustrates the main steps of a method according to the invention. First, attention is focused on the training phase, during which the latent spaces, as well as projection and backprojection functions, are defined. An important aspect of the invention is that the feature vector is projected not onto a single latent space, but onto several latent spaces defined during the training step. Thus, each feature, forming an input coordinate, is associated with a latent space, and preferably a single latent space. At least two different features of the feature vector are respectively projected into two different latent spaces.

The training phase comprises steps 100 to 160 shown in FIG. 2.

Step 100: Forming reference vectors.

During this step, a set of physiological data m_{i,j} measured on reference individuals refⱼ, forming a reference population, is available. The index i corresponds to a rank assigned to each physiological data point, with 1≤i≤I, I denoting the number of physiological data points measured. For each reference individual, based on the physiological data measured, features x_{i,j} are established, forming a feature vector Xⱼ defined in an input space (or real space) R. In this example, each feature is a measured physiological data point that is standardized (zero-mean, unit-variance):

$${x}_{i , j} = \frac{{m}_{i , j} - {\overline{m}}_{i}}{{σ}_{i}} \qquad ( 1 )$$

where m̅ᵢ is an average of the values m_{i,j} for all individuals J and σᵢ is the standard deviation of the features x_{i,j} of the same rank i.

In this example, the number of features corresponds to the number of physiological data measured.

The features of each reference individual form a reference vector Xⱼ, of dimension I, assigned to the individual refⱼ. Each feature x_{i,j} corresponds to an input coordinate of the reference individual refⱼ in the input space R.

For example, each measured value m_{i,j} may be an analysis of a parameter resulting from a blood test. For each reference individual refb, a reference vector Xⱼ is established comprising the measured values for the reference individual, standardized taking into account the entire reference population.

Steps 110 and 150 are then performed iteratively. k refers to a rank assigned to each iteration, with 1≤k≤K and K corresponding to the number of iterations.

### Step 110: Dimension Reduction

During this step, a dimensionality reduction algorithm is applied to all or part of the reference features of the reference vectors X_{j,k}. During the first iteration (k=1), the algorithm is applied to all the I features forming each reference vector: X_{j,k=1}=Xⱼ. During subsequent iterations, a reference vector X_{j,k} is used, which includes features x_{i,j} from the vector Xⱼ that were not selected during the previous iteration.

The dimensionality reduction algorithm defines a linear or nonlinear projection function ƒₖ. An example of this is a UMAP-type algorithm.

The projection function ƒₖ allows the vector X_{j,k} to be projected into a latent space Eₖ. In the latent space Eₖ, a projected vector V_{j,k}=fₖ (X_{j,k}) is obtained, whose dimension is less than the dimension of the vector X_{j,k}. The latent space Eₖ is usually referred to as a “manifold” by experts in the field.

The dimension of the projected vector V_{j,k} is usually between 1 and 10. Each term v_{h,j,k}, of the projected vector is a coordinate of the reference individual refⱼ in the latent space. The index h corresponds to a rank assigned to each term v_{h,j,k}. h is an integer between 1 and Hₖ, where Hₖ is the dimension of the latent space Eₖ. The dimension Hₖ is usually between 1 and 10.

### Step 120: Formation of the Latent Vector

The terms v_{h,j,k} of the projected vector V_{j,k} may form a latent vector Y_{j,k} of dimension Hₖ. According to one possibility, for each rank h, the terms v_{h,j,k} of the projected vector V_{j,k} may be standardized, so as to obtain standardized coordinates Y_{h,j,k} in the latent space

$${y}_{h , j , k} = \frac{{v}_{h , j , k} - {\overline{v}}_{h , k}}{{σ}_{h , k}} \qquad ( 2 )$$

Where v̅_{h,k} is an average of the terms v_{h,j,k} for all J reference individuals for the same rank h, and σ_{h,k} is the standard deviation of the terms v_{h,j,k} of the same rank h for all J reference individuals.

For each individual, the set of standardized coordinates y_{h,j,k} forms a latent vector Y_{j,k} of dimension Hₖ.

Thus, the latent vector Y_{j,k} contains either the terms v_{h,j,k} of the projected vector V_{j,k}, in which case Y_{j,k}=V_{j,k}, or the standardized terms y_{h,j,k} as described in connection with (2).

A backprojection (or regression) function

$${f}_{k}^{- 1}$$

may be used to estimate an input vector \hat{X}_{j,k} such that

$${\hat{X}}_{j , k} = {f}_{k}^{- 1} ( {Y}_{j , k} ) .$$

\hat{X}_{j,k} has the same dimension as X_{j,k}. Each term of the vector \hat{X}_{j,k} is an estimated feature \hat{x}_{i,j,k} in the input space R, based on the latent vector Y_{j,k}. The backprojection function ƒₖ⁻¹ may be established using a Nadaraya-Watson kernel estimator. Such an estimator is commonly used to establish nonparametric regression models. It is constructed from a kernel function K, for example Gaussian, and a window size h.

According to a variant, described below, a regression function

$${f}_{k , i}^{- 1}$$

is determined for each rank i. Thus, from the latent vector Y_{j,k}, a feature \hat{x}_{i,j,k} of rank i is estimated in the input space R such that:

$${\hat{x}}_{i , j , k} = {f}_{i , k}^{- 1} ( {Y}_{j , k} ) \qquad ( 3 )$$

$${f}_{i , k}^{- 1}$$

is a regression function determined for the feature of rank i. According to this variant, two regression functions, applied to the same latent vector and allowing the estimation of two different input coordinates, are different.

Step 130: Backprojection and estimation of the feature vector in the input space.

By applying the backprojection function

$${f}_{k}^{- 1}$$

to the latent vector Y_{j,k}, an estimate of a vector \hat{X}_{j,k}, defined in the input space R, is obtained, as described in step 110. \hat{X}_{j,k} is such that

$${\hat{X}}_{j , k} = {X}_{j , k} + {ε}_{j , k} \qquad ( 4 )$$

ϵ_{j,k} is a residual vector, with the same dimensions as \hat{X}_{j,k} and Xⱼ. ϵ_{j,k} corresponds to the residual from the model regression. The more representative the reference individual refⱼ is of the reference population, the lower the norm of ε_{j,k}.

The residual vector ε_{j,k} contains residuals ε_{i,j,k} associated with each feature of rank i:

$${\hat{x}}_{i , j , k} = {x}_{i , j , k} + {ε}_{i , j , k} \qquad ( 5 )$$

The distribution of ε_{i,j,k} values varies according to the reference individual refⱼ and the rank of the feature i.

The greater the dimension Hₖ, the lower the mean residual ε_{i,j,k} for each individual. It is considered that beyond a dimension of 3 or 4, the gain in terms of mean residual decreases or no longer changes significantly.

Each residual ε_{i,j,k} may be used to determine a z-score for individual i, relative to feature j, considering the latent space of rank k. The term “z-score” is a common term for professionals in the field.

### Step 140: Determination of a Relevance Indicator

During step 140, the features of rank i satisfying a selection criterion are selected. To this end, for each feature i, a relevance indicator is established that is representative of a statistical distribution of the values of the residual ε_{i,j,k}, for the said feature i and for all or some of the reference individuals.

The relevance indicator may be an average value

$${mean}_{j} ( {ε}_{i , j , k} )$$

considering the same rank i and all or part of the reference individuals. The relevance indicator may include the standard deviation of the residuals ε_{i,j,k}.

The relevance indicator may be representative of a dispersion of the residual ε_{i,j,k} considering the same rank i and all or part of the reference individuals. For example, it may be a variance or standard deviation σ_{ε}_{_{i,k}} of ε_{i,j,k} considering the different reference individuals.

### Step 150: Selection of Features

During this step, the relevance indicator resulting from step 140 is compared to a threshold. When the indicator is below the threshold, the latent space Eₖ is considered to be well suited to the feature i.

The threshold against which the dispersion indicator is compared may be a predetermined threshold or a threshold calculated by a known thresholding method, such as Otsu's thresholding.

When, for a feature of rank i, the selection criterion is met, i.e., when the statistical indicator such as σ_{ε}_{_{i,k}} or

$${mean}_{j} ( {ε}_{i , j , k} )$$

is below the threshold, the feature is considered to be well represented in the latent space Eₖ. In this case, the feature of rank i is associated with the latent space Eₖ.

Otherwise, the feature of rank i is not associated with the latent space Eₖ. A new iteration of steps 110 to 150 is implemented, of rank k+1. During the new iteration k+1, a reference vector X_{j,k+1} is used, which includes features x_{i,j} of the vector Xⱼ that were not selected during each previous iteration.

### Step 160

When, following the iterations of steps 110 and 150, each feature of rank i has been associated with a latent space Eₖ, the training phase is complete. For each input feature xᵢ:
one, and preferably only one, associated latent space Eₖ;
a projection function ƒₖ;
a backprojection function

$${f}_{k}^{- 1}$$

are defined.

The backprojection function is common to each input feature associated with the same latent space Eₖ. A backprojection function

$${f}_{i , k}^{- 1}$$

may be defined individually for each input feature associated with the same latent space Eₖ. In the latter case, for at least two features of different inputs, the backprojection function may be different.

The number K of iterations of steps 110 to 150 corresponds to the number of latent spaces Eₖ created.

### Variant

The reference population is assumed to consist of reference individuals in a reference state. In this example, the reference state is a non-pathological state. However, some reference individuals may be “false negatives,” i.e., reference individuals mistakenly considered to be in the reference state. In this example, these are unhealthy individuals, or healthy individuals whose physiological measurements have been affected by a bias, such as a measurement error. In order to avoid such a situation, the method may include, during the first iteration of steps 110 to 150 (k=1), a step 125 of filtering the reference population. During this step, standardized coordinate vectors Y_{j,k=1} are available for each reference individual refb. It is assumed that:
the reference population is likely to include a small number of individuals who are incorrectly considered to be part of the reference population. The number of such individuals is preferably considered to be less than 10% or 5% of the total population J.
the vectors of the standardized coordinates Y_{j,k=1} of the reference individuals, true negatives, are closer to each other than the vectors Y_{j,k=1} of false positive individuals.

The vectors Y_{j,k=1} are assumed to be distributed according to the same probability law. The probability density followed by the vectors may be estimated using a KDE (Kernel Density Estimation) method. This makes it possible to determine the probability density, of dimension Hₖ₌₁, of the vector Yₖ₌₁, the latter being considered as a random variable. The probability density is determined from the sample consisting of the vectors Y_{j,k=1} of the reference individuals. The kernel parameterizing the KDE method is predetermined. It can, for example, be considered Gaussian.

From among all the vectors Y_{j,k=1}, the n % most representative of the probability distribution thus determined, are then selected. n can, for example, be equal to 50% or 80% or more. The vectors Y_{j,k=1} thus selected are considered true negatives, i.e. corresponding to healthy reference individuals refⱼ, who are not affected by measurement errors. The unselected vectors Y_{j,k=1} are rejected. Step 100 is resumed, using only the selected reference individuals. The selection is performed in the latent space Eₖ₌₁. Advantage is taken of the low dimension Hₖ₌₁ of this latent space, compared to the dimension of the input space R. This facilitates the determination of the probability law followed by the different coordinates and the filtering of the reference population.

Although described with latent vectors, comprising the coordinates v_{i,j,k=1} or the standardized coordinates y_{i,j,k=1}, filtering step 125 may be implemented using the projected vector V_{j,k=1} instead of the latent vector Y_{j,k=1}, the projected vector comprising scaled but non-centered values.

This variant may be implemented independently of the consideration of different latent spaces. It may be implemented based on an estimation of the features from a single latent space, common to all the features.

Steps 200 to 240 describe the implementation of a method for processing features of a test individual that differ from those of the reference individuals.

The test individual is assigned a vector of physiological data m_{i,test}, of the same dimension as the vectors of physiological data m_{i,j} of the reference individuals. It is understood that for each rank, the physiological data of the test individual are of the same nature as the physiological data established for each reference individual.

Step 200: For each test subject, based on the measured physiological data m_{i,test}, features x_{i,test} are established, forming a feature vector Xₜₑₛₜ defined in the input space R. In this example, each feature is a measured physiological data point that has been standardized:

$${x}_{i , test} = \frac{{m}_{i , test} - {\overline{m}}_{i}}{{σ}_{i}} \qquad ( 6 )$$

m̅ᵢ and σᵢ were defined in step 100. When step 125 of filtering the reference individuals was implemented, the quantities m̅ᵢ and σᵢ are calculated from the selected reference individuals.

Step 210: projection into the K latent spaces Eₖ.

During this step, based on the vector Xₜₑₛₜ, K different vectors X_{test,k} are formed, K corresponding to the number of latent spaces Eₖ. Each vector X_{test,k} is projected into a latent space Eₖ in order to obtain as many projected vectors V_{test,k}=fₖ(X_{test,k}).

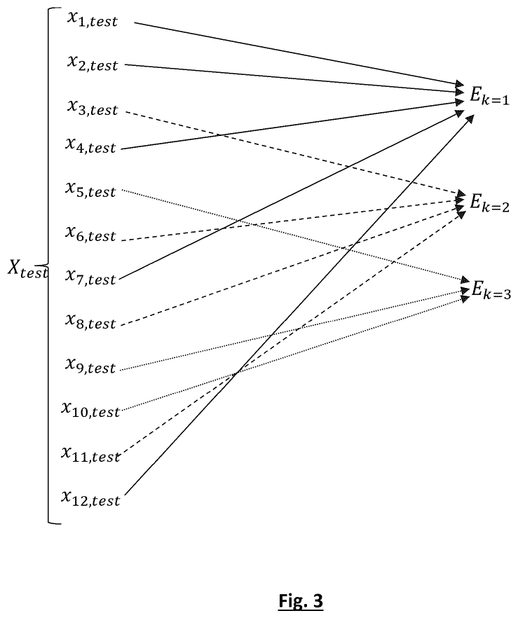

FIG. 3 shows an example in which the vector Xₜₑₛₜ has 12 features x_{i,test} . . . x_{12,test}. The features x_{i,test}, x_{2,test}, X_{4,test}, X_{7,test}, x_{12,test} form a vector X_{test,1}, be projected into a latent space Eₖ₌₁. The features x_{3,test}, x_{6,test}, x_{8,test}, x_{11,test} form a vector X_{test,2} which is projected into a latent space Eₖ₌₂. The features x_{5,test}, x_{9,test}, x_{10,test} form a vector X_{test,3} which is projected into a latent space Eₖ₌₃.

### Step 220: Obtaining Latent Coordinates

The projected vectors may be standardized in order to obtain standardized coordinates y_{h,test,k} in the latent space:

$${y}_{h , test , k} = \frac{{v}_{h , test , k} - {\overline{v}}_{h , k}}{{σ}_{h , k}} \qquad ( 7 )$$

Where v̅_{h,k} is an average of the terms v_{h,j,k} for all J reference individuals for the same rank h, and σ_{h,k} is the standard deviation of the terms v_{h,j,k} of the same rank h for all J reference individuals. When step 125 has been implemented, only the reference individuals selected during this step are taken into account.

The coordinates y_{h,test,k} form a vector Y_{test,k} of dimension Hₖ in each latent space Eₖ.

Alternatively, the projected vectors are not standardized. Each latent vector corresponds to a projected vector Y_{test,k}=V_{test,k}.

### Step 230: Backprojection

During step 230, the regression function

$${f}_{k}^{- 1}$$

corresponding to the latent space Eₖ is applied to the vector Y_{test,k} to estimate a vector \hat{X}_{test,k} such that

$${\hat{X}}_{t e st , k} = {f}_{k}^{- 1} ( {Y}_{t e st , k} )$$

where each term of the vector \hat{X}_{test,k} is an estimated feature \hat{x}_{i,test,k}, in the input space R, from the vectorY_{test,k} of standardized coordinates in the latent space Eₖ.

According to one possibility, each feature \hat{x}_{i,test,k} is such that

$${\hat{x}}_{i , test , k} = {f}_{i , k}^{- 1} ( {Y}_{test , k} )$$

Step 230 is performed from each latent space Eₖ to the input space R.

### Step 240: Calculation of a Score

During this step, a score zᵢ, usually referred to as a “z-score,” is determined for each feature of rank i, such that:

$${z}_{i , test} = \frac{{x}_{i , test} - {\hat{x}}_{i , test , k}}{{σ}_{{ε}_{i , k}}} = \frac{{ε}_{i , test}}{{σ}_{{ε}_{i , k}}} \qquad ( 8 )$$

In general, the z-score involves a comparison between x_{i,test} and \hat{x}_{i,test,k}. In the example of formula (8), the comparison is a subtraction normalized by the standard deviation σ_{ε}_{_{i,k}} defined in step 140.

The z-score z_{i,test} may be used to detect the presence of an anomaly affecting the feature x_{i,test} of rank i. When the absolute value of the z-score z_{i,test} is greater than a predetermined threshold value, the feature of rank i is considered abnormal. When the z-score is defined according to expression (8), and assuming that it is distributed according to a Gaussian law, the threshold value may be 1.96. Thus, when |z_{i,test}|≥1.96, the feature of rank i is considered abnormal. Thus, when −1.96<z_{i,test}<+1.96, the feature x_{i,test} of rank i is considered normal.

According to one possibility, the z-score z_{i,test} is determined according to the expression:

$${z}_{i , test} = \frac{{x}_{i , test} - {\hat{x}}_{i , test , k}}{{σ}_{{ε}_{i , k}}^{′}} \qquad ( 9 )$$

σ′_{ε}_{_{i,k}} is determined according to a procedure known as “leave-one-out,” in which iterative steps 110 and 150 are performed by successively removing a reference individual of rank j′. This makes it possible to form J sets of K latent spaces E_{k,j′}. Each latent space E_{k,j′} is defined from the reference population, without taking into account the reference individual of rank j′.

Subsequently, steps 200 to 230 are implemented using the individual of rank j′ as the test individual. Thus, for each individual of rank j′, a residual ε_{i,j′,k} is obtained such that ε_{i,j′,k}=x_{i,j′}−\hat{x}_{i,j′,k} (10).

σ_{ε}_{_{i,k}} corresponds to the standard deviation of the residuals ε_{i,j′,k} corresponding respectively to the different individuals of rank j′ considered.

According to one variant, the method takes into account a feature known as the contextual feature of the individual. The reference individuals are those in the reference population that have the said contextual feature. Contextual feature refers to the age and/or sex of the individual and/or weight. Steps 100 to 150 are performed by selecting reference individuals with the same contextual feature as the test individual.

### Experimental Tests.

In an initial series of tests, the physiological data determined were the volumes of brain structures observed on T1-weighted anatomical MRI images. Values representative of the asymmetry of brain structures between the right and left hemispheres were also determined. The feature vector for each individual had 187 components, corresponding to the dimension of the input space R.

The asymmetry of brain structures is quantified by an asymmetry coefficient, usually referred to as the “symmetry index” by professionals. This is a value between −1 and 1, calculated according to the expression

$${x}_{i} = \frac{{LV}_{i} - {RV}_{i}}{{LV}_{i} + {RV}_{i}}$$

where:
xᵢ=asymmetry coefficient of a brain structure of rank i;
LVᵢ: left volume of the brain structure of rank i;
RVᵢ=right volume of brain structure i

The asymmetry coefficient xᵢ was calculated for the identified brain structures.

The implementation of steps 110 to 150 led to the obtaining of four different latent spaces Eₖ₌₁, Eₖ₌₂, Eₖ₌₃ and Eₖ₌₄. The latent spaces Eₖ₌₁, Eₖ₌₂, Eₖ₌₃ and Eₖ₌₄ corresponded respectively to features:
volumes of white matter structures, basal ganglia, and ventricles;
volumes of gray matter structures;
volumes of other gray matter structures;
asymmetry data for different brain structures.

Steps 110 to 150 yielded four different latent spaces. The anatomical regions are distributed across the latent spaces as shown in Table 1.

TABLE 1

| k = 1 | k = 2 | k = 3 | k = 4 |
|---|---|---|---|
| Right nucleus | Right amygdala | Symmetry of the | Symmetry of the |
| accumbens | | hippocampus | nucleus accumbens |
| Left nucleus | Left amygdala | Symmetrical inferior | Symmetry of the |
| accumbens | | lateral ventricle | caudate nuclei |
| Brain stem | Right anterior | Symmetry of lateral | Symmetry of |
| | insula | ventricle | cerebellar white |
| | | | matter |
| Right caudate | Left anterior insula | Symmetry of the | Symmetry of |
| nucleus | | anterior cingulate | cerebral white |
| | | gyrus | matter |
| Left caudate | Right anterior | Symmetry of the | Symmetry of the |
| nucleus | orbital gyrus | anterior orbital gyrus | pallidum |
| Right cerebellar | Left anterior orbital | Symmetry of the | Symmetry of the |
| white matter | gyrus | cuneus | putamen |
| White matter of | Right angular | Symmetry of the | Symmetry of the |
| the left | gyrus | frontal operculum | thalamus |
| cerebellum | | | |
| Right cerebral | Left angular gyrus | Symmetry of the | Symmetry of the |
| white matter | | fusiform gyrus | ventral |
| | | | diencephalon |
| Left cerebral | Right calcarine | Symmetry of the | Symmetry of the left |
| white matter | cortex | rectus gyrus | telencephalon |
| Right | Left calcarine | Right inferior | Symmetry of the |
| hippocampus | cortex | temporal gyrus | insula |
| Left | Right cuneus | Left inferior temporal | Angular gyrus |
| hippocampus | | gyrus | symmetry |
| Right inferior | Left cuneus | Symmetry of the | Symmetry of the |
| lateral ventricle | | inferior temporal | calcarine cortex |
| | | gyrus | |
| Left inferior | Right entorhinal | Symmetry of the | Symmetry of the |
| lateral ventricle | cortex | medial frontal cortex | central operculum |
| Right lateral | Left entorhinal | Symmetry of the | Symmetry of the |
| ventricle | cortex | middle frontal gyrus | entorhinal cortex |
| Left lateral | Right frontal | Symmetry of the | Symmetry of the |
| ventricle | operculum | middle occipital gyrus | frontal pole |
| Right pallidum | Left frontal | Medial segment of | Symmetry of the |
| | operculum | the right precentral | lingual gyrus |
| | | gyrus | |
| Left pallidum | Right frontal pole | Medial segment of | Symmetry of the |
| | | the left precentral | lateral orbital gyrus |
| | | gyrus | |
| Right putamen | Left frontal pole | Symmetry of the | Symmetry of the |
| | | middle segment of | medial orbital gyrus |
| | | the precentral gyrus | |
| Left putamen | Right fusiform | Median segment of | Symmetry of the |
| | gyrus | the right superior | middle segment of |
| | | frontal gyrus | the postcentral |
| | | | gyrus |
| Right thalamus | Left fusiform gyrus | Symmetrical middle | Symmetry of the |
| | | temporal gyrus | occipital pole |
| Left thalamus | Right inferior | Right fusiform | Symmetry of the |
| | occipital gyrus | occipital gyrus | inferior orbital |
| | | | frontal gyrus |
| Right ventral | Left inferior | Left fusiform occipital | Symmetry of the |
| diencephalon | occipital gyrus | gyrus | precuneus |
| Left ventral | Symmetry of the | Symmetry of the | Symmetry of the |
| diencephalon | inferior occipital | fusiform occipital | parahippocampal |
| | gyrus | gyrus | gyrus |
| Right forebrain | Right lingual gyrus | Symmetry of the | Symmetry of the |
| | | inferior frontal | posterior insula |
| | | opercular gyrus. | |
| Left forebrain | Left lingual gyrus | Right posterior | Symmetry of the |
| | | cingulate gyrus | postcentral gyrus |
| Right anterior | Right lateral orbital | Left posterior | Symmetry of the |
| cingulate gyrus | gyrus | cingulate gyrus | posterior orbital |
| | | | gyrus |
| Left anterior | Left lateral orbital | Symmetry of the | Symmetry of the |
| cingulate gyrus | gyrus | posterior cingulate | polar plane |
| | | gyrus | |
| Right central | Right middle | Right | Symmetry of the |
| operculum | cingulate gyrus | parahippocampal | precentral gyrus |
| | | gyrus | |
| Left central | Left middle | Left parahippocampal | Symmetry of the |
| operculum | cingulate gyrus | gyrus | subcallosal zone |
| Right rectus | Symmetry of the | Symmetry of the | Right |
| gyrus | middle cingulate | superior frontal | supplementary |
| | gyrus | gyrus. | motor cortex |
| Left rectus gyrus | Right middle | | Left supplementary |
| | occipital gyrus | | motor cortex |
| Right medial | Left middle | | Symmetry of the |
| frontal cortex | occipital gyrus | | supramarginal gyrus |
| Left medial | Right medial orbital | | |
| frontal cortex | gyrus | | superior occipital |
| | | | gyrus |
| Right middle | Left medial orbital | | Symmetry of the |
| frontal gyrus | gyrus | | superior parietal |
| | | | lobule |
| Left middle | Medial segment of | | Symmetry of the |
| frontal gyrus | the right | | superior temporal |
| | postcentral gyrus | | gyrus |
| Median right | Median segment of | | Symmetry of the |
| superior frontal | the left postcentral | | temporal pole |
| gyrus | gyrus | | |
| Medial left | Right middle | | Symmetry of the |
| superior frontal | temporal gyrus | | inferior frontal gyrus |
| gyrus | | | |
| Left precuneus | Left middle | | |
| | temporal gyrus | | |
| Right precuneus | Right occipital pole | | |
| Right | Left occipital pole | | |
| subcallosal zone | | | |
| Left subcallosal | Right inferior | | |
| area | frontal opercular | | |
| | gyrus | | |
| Right superior | Left inferior frontal | | |
| frontal gyrus | opercular gyrus | | |
| Left superior | Right inferior | | |
| frontal gyrus | orbital frontal gyrus | | |
| Right superior | Right frontal | | |
| temporal gyrus | inferior orbital | | |
| | gyrus | | |
| Left superior | Left inferior frontal | | |
| temporal gyrus | orbital gyrus | | |
| Right temporal | Right posterior | | |
| pole | insula | | |
| Left temporal | Left posterior | | |
| pole | insula | | |
| Right triangular | Right parietal | | |
| inferior frontal | operculum | | |
| gyrus | | | |
| Left triangular | Left parietal | | |
| inferior frontal | operculum | | |
| gyrus | | | |
| | Symmetry of the | | |
| | parietal operculum | | |
| | Right postcentral | | |
| | gyrus | | |
| | Left postcentral | | |
| | gyrus | | |
| | Right posterior | | |
| | orbital gyrus | | |
| | Left posterior | | |
| | orbital gyrus | | |
| | Right polar plane | | |
| | Left polar planum | | |
| | Right precentral | | |
| | gyrus | | |
| | Left precentral | | |
| | gyrus | | |
| | Right temporal | | |
| | planum | | |
| | Left temporal | | |
| | planum | | |
| | Symmetry of the | | |
| | planum temporale | | |
| | Symmetry of the | | |
| | supplementary | | |
| | motor cortex | | |
| | Right | | |
| | supramarginal | | |
| | gyrus | | |
| | Left supramarginal | | |
| | gyrus | | |
| | Right superior | | |
| | occipital gyrus | | |
| | Left superior | | |
| | occipital gyrus | | |
| | Right superior | | |
| | parietal lobule | | |
| | Left superior | | |
| | parietal lobule | | |
| | Right transverse | | |
| | temporal gyrus | | |
| | Left transverse | | |
| | temporal gyrus | | |
| | Symmetry of the | | |
| | transverse | | |
| | temporal gyrus | | |

It is observed that each latent space corresponds either to an anatomical region or to a type of feature (volumes for spaces Eₖ₌₁, Eₖ₌₂, Eₖ₌₃, asymmetry coefficient for space Eₖ₌₄). This demonstrates the relevance of the approach, which defines several latent spaces and to associate each feature with one of the defined latent spaces.

In another series of tests, the data analyzed were cortical thicknesses, determined by T1-weighted MRI. The feature vector for each individual had 98 components, corresponding to the dimension of the input space R. A population of 3,187 different individuals was taken into account. Each component corresponded to a cortical thickness of an anatomical region of the cortex, which had been segmented into 98 regions.

A pathological cohort of 28 epileptic subjects was available, all of whom had a hypertrophic region linked to epilepsy, which had been identified and treated surgically. A control cohort consisted of 30 control subjects, considered to be normal.

Each subject underwent an examination in order to extract a feature vector corresponding to the volume of each of the 98 anatomical regions mentioned above. For each subject in each cohort, the following was implemented:
a method of analyzing the prior art, using the “Lifespan” algorithm, based on charts defining a normal range of anatomical volume according to age. The Lifespan algorithm was described in the publication by Coupé et al. “Alzheimer's disease neuroimaging initiative. Towards a unified analysis of brain maturation and aging across the entire lifespan; A MRI analysis”; Hum Brain Mapp 38,5501-5518 (2017).
the method described in connection with steps 200 to 250 (invention).

The following was observed:
for subjects in the pathological cohort, 60% of atypical epileptic cortical regions (atrophic or hypertrophic) when implementing the invention, compared to 39% when implementing the prior art method;
for subjects in the control cohort, 7±3 atypical cortical regions, compared to 11±5 when implementing the prior art method.

These results show that, compared to the prior art, implementation of the method is accompanied by improved sensitivity (see results obtained on the pathological cohort) and improved specificity (see results from the control cohort).

Furthermore, when implementing steps 200 to 250, a backprojection function

$${f}_{i , k}^{- 1}$$

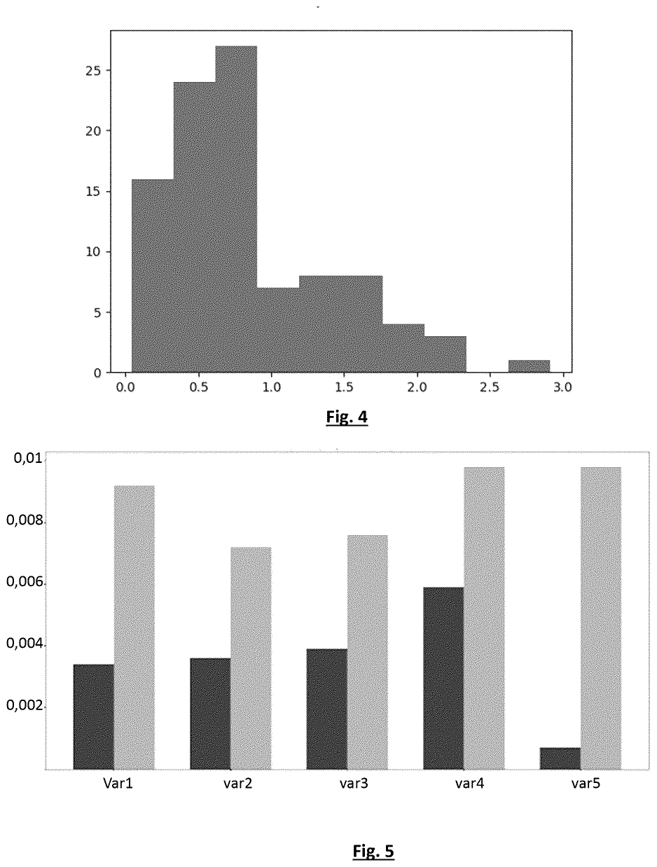

defined individually for each feature was used during backprojection. Each backprojection function was a Nadaraya-Watson estimator based on a Gaussian kernel weighting function. FIG. 4 shows a histogram of the values of the estimator window size h for each of the 98 features of rank i. It can be seen that the size is distributed between 0.1 and 2.8. By imposing the same regression function for all features, the size was 1.67. Having a degree of freedom in terms of kernel size allows to use an individualized regression function for each feature, with its own features, in this case the window size.

In another series of tests, data from blood counts resulting from laboratory analyses were used. Five features were considered: white blood cell count per 1 mL (var1), red blood cell count per 1 mL (var2), hemoglobin level expressed in g/dL (var3), hematocrit percentage (var4), and mean corpuscular volume expressed in fL (var5). Blood counts were available for 1,975 individuals.

FIG. 5 shows, for each feature studied (x-axis), the mean square error between the standardized feature xᵢ resulting from step 200 and its estimate \hat{x}_{i,k} calculated by regression. The mean square error corresponds to the y-axis. The estimate \hat{x}_{i,k} was calculated:
either by implementing an identical regression mode

$${f}_{k}^{- 1}$$

for each feature (gray vertical bars in FIG. 5)
or by implementing an individualized regression model

$${f}_{i , k}^{- 1}$$

for each feature (black vertical bars in FIG. 5)

It is observed that the mean square error is systematically lower when using an individualized regression model.

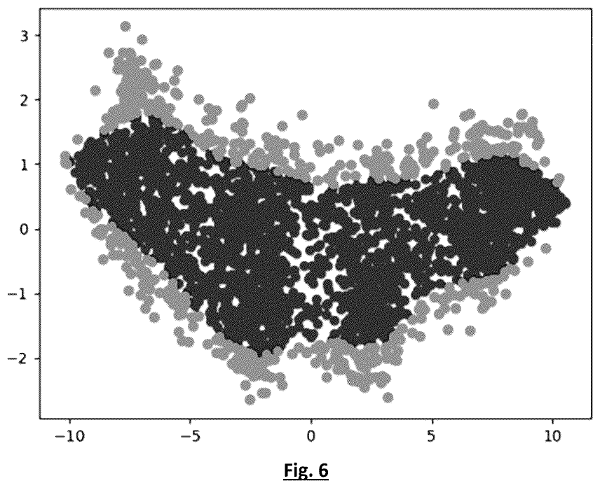

In another series of tests, blood ionograms were taken into account. 28 ion concentrations were available for a population of 4,188 individuals. FIG. 6 shows the coordinates of each feature along two axes of the rank-1 latent space Eₖ₌₁. These coordinates are obtained during the first iteration of step 120.

Step 125 described above was then implemented. From the set of vectors Y_{j,k=1}, the 70% most representative of the probability distribution followed by the variables in the latent space were selected. FIG. 6 illustrates this filtering: the black dots correspond to the unfiltered latent variables. The gray dots correspond to the filtered latent variables.

The invention may be implemented using physiological data resulting from different types of analysis for diagnostic assistance purposes.

1. Method for processing physiological data from an individual, each physiological data point resulting from measurements taken on the individual, the method comprising:
a) based on the physiological data of the individual, assigning coordinates to the individual, known as input coordinates, defined in an input space, with each of the input coordinates being assigned a rank;
b) associating each input coordinate of the individual, in the input space, with a latent space of a dimension lower than a dimension of the input space, the associating of each of the input coordinates with the latent space resulting from a training phase;
c) projecting each of the input coordinates resulting from the assigning a) into the latent space associated with the input coordinate, so as to obtain a latent coordinate of the individual in the latent space, and forming a latent vector in the latent space associated with the input coordinate;
d) applying a regression function to the latent vector resulting from the projecting c), so as to estimate an input coordinate in the input space for each rank, the regression function being associated with the latent space associated with the input coordinate, the regression function being defined during the training phase; and
e) for at least one rank, comparing the input coordinates of the individual resulting from the applying d) and the assigning a) and determining a score for the individual based on the comparison;
wherein
in the associating b), the respective latent space associated with each of the input coordinates belongs to a set of latent spaces, the association of each of the input coordinates with respective the latent space being performed according to the rank of the input coordinate;
in the projecting c), the latent coordinates in the same latent space form a latent vector; and
the applying d) comprises applying the regression function to each latent vector resulting from the projecting c);
and wherein each latent space is determined from physiological data established on reference individuals from a reference population, according to the training phase, wherein the training phase comprises:
(i) for each reference individual, based on measured physiological data, assigning reference coordinates in the input space;
(ii) applying a dimension reduction algorithm to the reference coordinates of each reference individual, so as to define, for each reference individual, latent coordinates in a latent space, the applying (ii) being implemented, during a first iteration, for all ranks and, during subsequent iterations, for ranks not selected in the selecting v) of a previous iteration, the latent coordinates in the same latent space forming a latent vector;
(iii) applying a regression function to each latent vector, so as to obtain an estimate of the input coordinate for each rank in the input space;
(iv) comparing the input coordinates of each reference individual resulting from the assigning (i) and the applying (iii) in order to obtain, for each rank, a relevance indicator;
(v) selecting ranks for which the relevance indicator satisfies the selection condition; and
(vi) repeating the applying (ii), the applying (iii), the comparing (iv) and the selecting v) for the ranks not selected in the selecting v), the repeating (vi) being carried out until an iteration termination criterion is reached.

2. The method according to claim 1, wherein the projecting c) comprises applying a regression function to each latent vector in the latent space, wherein two regression functions, applied to the same latent vector and allowing the estimation of two different input coordinates, are different.

3. The method according to claim 1, wherein, during the first iteration, the applying (ii) comprises:
(ii-a) based on the coordinates of the reference individuals in the latent space, selecting reference individuals representative of the reference population; and
(ii-b) repeating the assigning (i) and the applying (ii) without taking into account the reference individuals selected during the repeating (ii-b).

4. The method according to claim 3, wherein the selecting (ii-a) comprises:
estimating a probability density from the latent vectors respectively associated with each reference individual;
for each reference individual, calculating a probability density value from the latent vector associated with the reference individual; and
selecting the reference individuals for which the probability density value is greater than a predetermined threshold.

5. The method according to claim 1, comprising, prior to the assigning a), obtaining physiological data from the individual using at least one sensor.

6. The method according to claim 5, comprising
forming a two-dimensional or three-dimensional image of the individual; and
extracting physiological data from each image of the individual.

7. The method according to claim 6, wherein the image results from an imaging modality.

8. The method according to claim 5, wherein the physiological data results from an analysis modality.

9. The method according to claim 5, comprising performing an analysis of a bodily fluid of the user, previously collected, the physiological data of the individual resulting from the analysis of the bodily fluid.

10. A system for determining a condition of an individual, the system comprising:
a sensor, or a set of sensors, configured to measure physiological data of the individual;
a processing unit configured to receive the physiological data measured by the sensor or the set of sensors, and to implement a method comprising:
a) based on the physiological data of the individual, assigning coordinates to the individual, known as input coordinates, defined in an input space, with each of the input coordinates being assigned a rank;
b) associating each input coordinate of the individual, in the input space, with a latent space of a dimension lower than a dimension of the input space, the associating of each of the input coordinates with the latent space resulting from a training phase;
c) projecting each of the input coordinates resulting from the assigning a) into the latent space associated with the input coordinate, so as to obtain a latent coordinate of the individual in the latent space, and forming a latent vector in the latent space associated with the input coordinate;
d) applying a regression function to the latent vector resulting from the projecting c), so as to estimate an input coordinate in the input space for each rank, the regression function being associated with the latent space associated with the input coordinate, the regression function being defined during the training phase; and
e) for at least one rank, comparing the input coordinates of the individual resulting from the applying d) and the assigning a) and determining a score for the individual based on the comparison;
wherein
in the associating b), the respective latent space associated with each of the input coordinates belongs to a set of latent spaces, the association of each of the input coordinates with respective the latent space being performed according to the rank of the input coordinate;
in the projecting c), the latent coordinates in the same latent space form a latent vector; and
the applying d) comprises applying the regression function to each latent vector resulting from the projecting c);
and wherein each latent space is determined from physiological data established on reference individuals from a reference population, according to the training phase, wherein the training phase comprises:
(i) for each reference individual, based on measured physiological data, assigning reference coordinates in the input space;
(ii) applying a dimension reduction algorithm to the reference coordinates of each reference individual, so as to define, for each reference individual, latent coordinates in a latent space, the applying (ii) being implemented, during a first iteration, for all ranks and, during subsequent iterations, for ranks not selected in the selecting v) of a previous iteration, the latent coordinates in the same latent space forming a latent vector;
(iii) applying a regression function to each latent vector, so as to obtain an estimate of the input coordinate for each rank in the input space;
(iv) comparing the input coordinates of each reference individual resulting from the assigning (i) and the applying (iii) in order to obtain, for each rank, a relevance indicator;
(v) selecting ranks for which the relevance indicator satisfies the selection condition; and
(vi) repeating the applying (ii), the applying (iii), the comparing (iv) and the selecting v) for the ranks not selected in the selecting v), the repeating (vi) being carried out until an iteration termination criterion is reached.

11. The method according to claim 7, wherein the imaging modality is selected from the group consisting of MRI, computed tomography, and scintigraphy.

12. The method according to claim 8, wherein the analysis modality is selected from the group consisting of electrocardiogram, electromyogram, electroencephalogram, magnetoencephalography, oximetry, and body gas composition analysis.

13. The method according to claim 5, wherein the physiological data of the individual results from an analysis of a bodily fluid of the user.

14. The method according to claim 13, wherein the bodily fluid of the user was collected prior to the analysis.

15. The method according to claim 2, wherein, during the first iteration, the applying (ii) comprises:
(ii-a) based on the coordinates of the reference individuals in the latent space, selecting reference individuals representative of the reference population; and
(ii-b) repeating the assigning (i) and the applying (ii) without taking into account the reference individuals selected during the repeating (ii-b).

16. The method according to claim 15, wherein the selecting (ii-a) comprises:
estimating a probability density from the latent vectors respectively associated with each reference individual;
for each reference individual, calculating a probability density value from the latent vector associated with the reference individual; and
selecting the reference individuals for which the probability density value is greater than a predetermined threshold.

17. The method according to claim 2, comprising, prior to the assigning a), obtaining physiological data from the individual using at least one sensor.

18. The method according to claim 17, comprising
forming a two-dimensional or three-dimensional image of the individual; and
extracting physiological data from each image of the individual.

19. The method according to claim 18, wherein the image results from an imaging modality.

20. The method according to claim 17, wherein the physiological data results from an analysis modality.
